(19) United States
(12) Patent Application Publication
(10) Pub. No.: US 2023/0248468 A1
(43) Pub. Date: Aug. 10, 2023

(54) MEDICAL DISPLAY SYSTEM, CONTROL METHOD, AND CONTROL DEVICE

(71) Applicant: SONY GROUP CORPORATION, TOKYO (JP)

(72) Inventors: KANA MATSUURA, TOKYO (JP); SHINJI KATSUKI, TOKYO (JP); TAKESHI MAEDA, TOKYO (JP)

(21) Appl. No.: 18/004,688

(22) PCT Filed: Jul. 2, 2021
(86) PCT No.: PCT/JP2021/025079

§ 371 Date: Jan. 9, 2023

(30) Foreign Application Priority Data
Jul. 16, 2020 (JP) 2020-122015

(51) Int. Cl. A61B 90/00; A61B 34/10; G06F 3/01; G10L 15/22; G10L 17/00

(52) U.S. Cl. CPC A61B 90/37; A61B 34/10; A61B 90/361; G06F 3/013; G10L 15/22; G10L 17/00; G10L 2015/223

(57) ABSTRACT

The present technology relates to a medical display system, a control method, and a control device which make it possible to prevent an unintended operation. Provided is a medical display system including: a display unit that provides a display on the basis of information output from a medical device; an imaging unit that images, as an imaging region, a part of a region from which at least the display unit is viewable; a voice acquisition unit that acquires a voice in the region from which at least the display unit is viewable; and a control unit that controls display information on the basis of the information output from the medical device. When a first user registered in advance satisfies a predetermined condition in the imaging region, the control unit controls the display information on the basis of a voice of the first user or an input triggered by the voice, and when the first user does not satisfy the predetermined condition in the imaging region, the control unit controls the display information on the basis of a voice of a second user different from the first user or an input triggered by the voice.

### TECHNICAL FIELD

The present technology relates to a medical display system, a control method, and a control device, and particularly relates to a medical display system, a control method, and a control device which make it possible to prevent an unintended operation.

### BACKGROUND ART

An operating room is divided into several zones such as a clean area, an unclean area, and the like. For example, an operator such as a surgeon performs surgery in a clean area. At this time, the operator in the clean area can operate only medical devices satisfying a predetermined clean standard by hand in order to keep his/her hands clean.

In addition, it is difficult to perform detailed operation of a medical device using a foot pedal or the like with foot. In view of this, it has been proposed to operate a medical device also using a voice input as disclosed in Patent Document 1.

### CITATION LIST

### Patent Document

Patent Document 1: WO 2018/173681 A

### SUMMARY OF THE INVENTION

### Problems to be Solved by the Invention

Meanwhile, there are several medical workers such as an assistant, a nurse, and a clinical engineer in an operating room in addition to the operator as surgical participants. In addition, the operator may change to another doctor during the surgery. Therefore, when voice input is used to operate devices, it is required to prevent an unintended operation.

The present technology has been made in view of the above circumstances, and is intended to prevent an unintended operation.

### Solutions to Problems

A medical display system according to one aspect of the present technology includes: a display unit that provides a display on the basis of information output from a medical device; an imaging unit that images, as an imaging region, a part of a region from which at least the display unit is viewable; a voice acquisition unit that acquires a voice in the region from which at least the display unit is viewable; and a control unit that controls display information on the basis of the information output from the medical device, in which the control unit controls, when a first user registered in advance satisfies a predetermined condition in the imaging region, the display information on the basis of a voice of the first user or an input triggered by the voice, and controls, when the first user does not satisfy the predetermined condition in the imaging region, the display information on the basis of a voice of a second user different from the first user or an input triggered by the voice.

A control method according to one aspect of the present technology is a control method performed when a medical display system controls display information displayed on a display unit on the basis of information output from a medical device, the method including: controlling, when a first user registered in advance satisfies a predetermined condition in an imaging region that is imaged as a part of a region from which at least the display unit is viewable, the display information on the basis of a voice of the first user acquired in the region from which at least the display unit is viewable or an input triggered by the voice; and controlling, when the first user does not satisfy the predetermined condition in the imaging region, the display information on the basis of a voice of a second user acquired in the region from which at least the display unit is viewable or an input triggered by the voice, the second user being different from the first user.

During control of display information displayed on the display unit on the basis of information output from the medical device, the medical display system and the control method according to one aspect of the present technology controls, when a first user registered in advance satisfies a predetermined condition in the imaging region that is obtained by imaging a part of a region from which at least the display unit is viewable, the display information on the basis of a voice of the first user acquired in the region from which at least the display unit is viewable or an input triggered by the voice, and controls, when the first user does not satisfy the predetermined condition in the imaging region, the display information on the basis of a voice of a second user acquired in the region from which at least the display unit is viewable or an input triggered by the voice, the second user being different from the first user.

A control device according to one aspect of the present technology includes a control unit that controls display information displayed on a display unit on the basis of information output from a medical device, in which the control unit controls, when a first user registered in advance satisfies a predetermined condition in an imaging region that is imaged as a part of a region from which at least the display unit is viewable, the display information on the basis of a voice of the first user acquired in the region from which at least the display unit is viewable or an input triggered by the voice, and controls, when the first user does not satisfy the predetermined condition in the imaging region, the display information on the basis of a voice of a second user acquired in the region from which at least the display unit is viewable or an input triggered by the voice, the second user being different from the first user.

When the display information displayed on the display unit is controlled on the basis of information output from the medical device, and a first user registered in advance satisfies a predetermined condition in the imaging region that is obtained by imaging a part of a region from which at least the display unit is viewable, the control device according to one aspect of the present technology controls the display information on the basis of a voice of the first user acquired in the region from which at least the display unit is viewable or an input triggered by the voice, and when the first user does not satisfy the predetermined condition in the imaging region, the control device controls the display information on the basis of a voice of a second user acquired in the region from which at least the display unit is viewable or an input triggered by the voice, the second user being different from the first user.

Note that the control device according to one aspect of the present technology may be an independent device or an internal block constituting one apparatus.

### BRIEF DESCRIPTION OF DRAWINGS

FIG. 1 is a diagram illustrating a configuration example of a medical display system according to an embodiment.

FIG. 2 is a diagram illustrating an example of input/output and processing performed by a control device.

FIG. 3 is a diagram illustrating an example of voice activation in a case where there are multiple user candidates.

FIG. 4 is a flowchart for describing the flow of user determination processing when voice input is used.

FIG. 5 is a diagram illustrating an example of operation authority for each category.

FIG. 6 is a diagram illustrating an example of a doctor table.

FIG. 7 is a diagram illustrating an example of a preoperative registration table.

FIG. 8 is a diagram illustrating a specific example of user determination during surgery.

FIG. 9 is a flowchart for describing the flow of voice-enabled processing.

FIG. 10 is a flowchart for describing the flow of line-of-sight-enabled processing.

FIG. 11 is a flowchart for describing the flow of user determination processing according to rank.

FIG. 12 is a flowchart for describing the flow of authority transfer processing.

FIG. 13 is a flowchart for describing the flow of authority transfer cancellation processing.

FIG. 14 is a flowchart for describing the flow of user exclusion processing.

### MODE FOR CARRYING OUT THE INVENTION

### 1. First Embodiment

### System Configuration

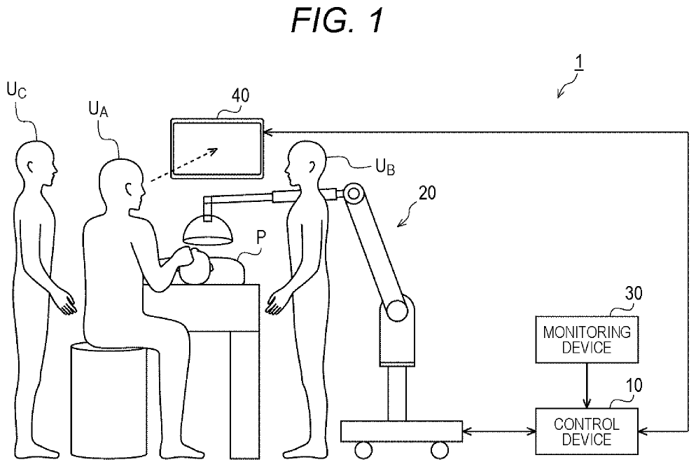

FIG. 1 illustrates a state of surgery using a medical display system according to an embodiment of the present technology.

In FIG. 1, a medical display system 1 includes a control device 10, a microscope device 20, a monitoring device 30, and a display device 40.

The example of FIG. 1 shows a state in which a user U_{A} as an operator is performing an operation on a patient P on a patient bed using the medical display system 1 that includes the above-mentioned devices. In addition, user candidates U_{B} and U_{C} as an assistant, a nurse, and the like are around the patient P on the patient bed.

In the following description, the “user” refers to a person having operation authority based on voice input such as the operator or the like. In addition, the “user candidate” includes a medical worker such as an operator, an assistant, a nurse, and a clinical engineer, and means any surgical staff (surgical participant) who uses the medical display system 1. That is, the user candidates as surgical staffs include the user having operation authority based on voice input.

The control device 10 is a device capable of controlling a connected device such as a medical device or integrating information output from the connected device, and is, for example, a camera control unit (CCU) or the like. The control device 10 may be connected to a network so as to be communicable with an external device such as a server or a personal computer (PC).

The microscope device 20 is an electron imaging microscope device (so-called video microscope device). The microscope device 20 captures an image of a surgical site of the patient P and outputs a signal representing an operative field image including the operative field to the control device 10. Note that, as a device that captures an operative field image, a medical imaging device such as an endoscope may be used instead of the microscope device 20.

The monitoring device 30 monitors biological information of the patient P and generates monitoring information indicating a monitoring result of the biological information. The biological information of the patient P includes a heart rate, an electrocardiogram, blood oxygen saturation, arterial pressure, and the like. The monitoring device 30 outputs a signal indicating the monitoring information to the control device 10.

The display device 40 is a device that displays information output from the control device 10, and is, for example, a liquid crystal display, an electro luminescence (EL) display, or the like. The display device 40 is provided near a user or a user candidate such as on a wall surface of the operating room. The display device 40 displays various types of information regarding the surgery such as biological information and physical information of the patient P and information about an operative method of the surgery together with the operative field image captured by the microscope device 20.

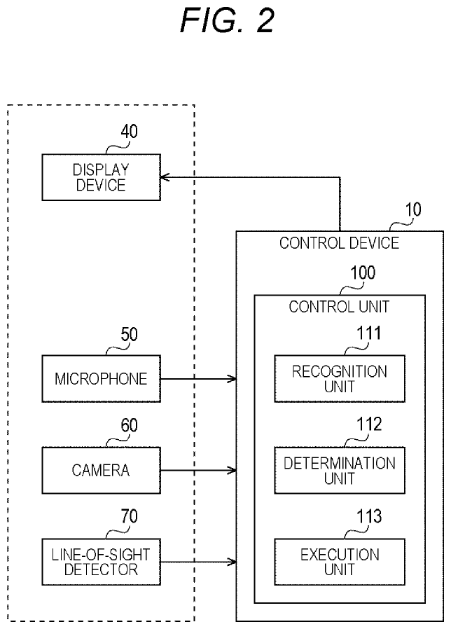

FIG. 2 is a diagram illustrating an example of input/output and processing performed by the control device 10 in FIG. 1.

As illustrated in FIG. 2, signals from a microphone 50, a camera 60, and a line-of-sight detector 70 are input to the control device 10.

The microphone 50 is a device capable of detecting the voice of a user candidate such as an operator, and is, for example, an array microphone. The microphone 50 outputs a signal (voice signal) representing the voice issued from the user candidate to the control device 10.

The camera 60 is a device that captures an image of a user candidate such as an operator, and is, for example, an operating room camera. The camera 60 outputs a signal (image signal) representing the captured image including the user candidate to the control device 10.

The line-of-sight detector 70 is a device that detects the line-of-sight of a user candidate such as an operator, and is, for example, an infrared (IR) camera. The line-of-sight detector 70 outputs a signal (detection signal) representing the line-of-sight of the user candidate to the control device 10.

The microphone 50, the camera 60, and the line-of-sight detector 70 may be mounted on the display device 40, or may be provided as an independent device and connected to the control device 10. Note that, although the example of FIG. 2 shows the configuration in which the line-of-sight detector 70 is provided as a device for detecting line-of-sight, the line-of-sight of the user candidate may be detected by analyzing a captured image captured by the camera 60.

The control device 10 acquires signals output from the microphone 50, the camera 60, and the line-of-sight detector 70. The control device 10 controls a medical device such as the microscope device 20 or controls display information displayed on the display device 40 on the basis of an analysis result of the acquired signals.

The control device 10 includes a control unit 100 including a recognition unit 111, a determination unit 112, and an execution unit 113.

The recognition unit 111 performs predetermined recognition processing on the basis of signals output from the microphone 50, the camera 60, and the line-of-sight detector 70, and supplies a recognition result to the determination unit 112.

For example, the recognition unit 111 recognizes a voice command included in the speech of the user candidate on the basis of the voice signal from the microphone 50. The recognition unit 111 also recognizes the user candidate on the basis of the voice signal from the microphone 50 or the image signal from the camera 60. The recognition unit 111 also recognizes the line-of-sight position of the user candidate on the basis of the image signal from the camera 60 or the detection signal from the line-of-sight detector 70.

The determination unit 112 determines whether or not the user candidate satisfies a predetermined condition for being a user having operation authority on the basis of the recognition result from the recognition unit 111, and supplies the determination result to the execution unit 113. The predetermined condition is determined using information acquired before the start of surgery, information acquired during surgery, and the like.

In a case where the determination result from the determination unit 112 satisfies the predetermined condition, the execution unit 113 executes predetermined processing on the basis of the user's voice or an input triggered by the voice.

For example, the voice of the user includes a voice command issued from the user. Furthermore, the input triggered by the voice includes the line-of-sight of the user when the user issues the voice command. The execution unit 113 controls the display information displayed on the display device 40 as predetermined processing in response to the voice command issued from the user on the basis of the line-of-sight position of the user on a screen of the display device 40.

Note that the processing executed by the control unit 100 may be executed by an external device other than the control device 10. Alternatively, some of the processes executed by the recognition unit 111, the determination unit 112, and the execution unit 113 in the control unit 100 may be executed by an external device.

As described above, in the medical display system 1, the display information generated by the control device 10 is displayed on the display device 40 during surgery. The user performs various treatments such as removal of an affected site while viewing the display information displayed on the display device 14 and observing the state of the surgical site. Furthermore, when performing various treatments, the user can operate each device constituting the medical display system 1 by his/her voice or line-of-sight.

### Example of Voice Activation

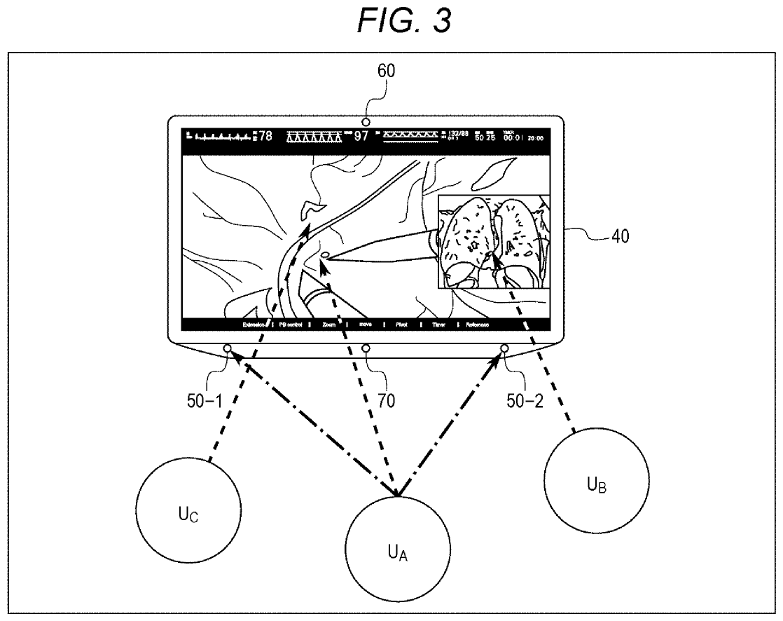

FIG. 3 illustrates an example of an operation using voice input in a case where there are multiple user candidates in the operating room.

In FIG. 3, the display device 40 displays display information including information necessary for surgery. In the example of FIG. 3, an operative field image, a reference image, monitoring information, an operation menu, and the like are displayed as the display information, and several user candidates each view these pieces of information. In FIG. 3, three circles in front of the screen of the display device 40 represent three user candidates U_{A} to U_{C}, respectively.

In addition, the display device 40 is equipped with the microphone 50, the camera 60, and the line-of-sight detector 70, and can detect voice input, line-of-sight, and the like from or of the user candidates U_{A} to U_{C}. In FIG. 3, the direction of the speech of the user candidate is indicated by a dash-dot-dash line, and the direction of the line-of-sight is indicated by a broken line.

At this time, the operation for changing (the content of) the display information displayed on the display device 40 is performed by voice input or the line-of-sight of the user candidate, or a combination thereof. However, a malfunction is caused in a case where the speech of the user candidate U_{B} is recognized as the voice activation in a situation where the user candidate U_{A} should perform the voice activation.

Furthermore, in a case where the user candidate U_{A} issues a voice command and at the same time, the line-of-sight position of the user candidate U_{B} is recognized as the operation under a situation where the operation is executed by a combination of a voice and a line-of-sight and the user candidate U_{A} is to perform the operation, an operation not intended by the user candidate U_{A} is caused.

Note that the user candidates U_{A} to U_{C} are recognized using the camera 60 capable of imaging, from the screen side, the user candidates U_{A} to U_{C} who are in a region from which the screen of the display device 40 is viewable, in addition to the microphone 50 and the line-of-sight detector 70 which are input devices for detecting an operation.

That is, the microphone 50 and the line-of-sight detector 70 can detect a voice and a line-of-sight in a region from which at least the screen of the display device 40 is viewable, and can detect voices issued by the three user candidates U_{A} to U_{C} and lines-of-sight of the three user candidates U_{A} to U_{C}, respectively. Furthermore, the camera 60 can capture a part of the region from which at least the screen of the display device 40 is viewable as an imaging region, and in the example of FIG. 3, three user candidates U_{A} to U_{C} are within the imaging region.

As described above, there are several medical workers such as an assistant, a nurse, and a clinical engineer in the operating room in addition to the operator as user candidates. Therefore, when voice activation is used, it is required to extract a speech including a voice command of an appropriate user from among the plurality of user candidates to prevent an unintended operation. In addition, it is necessary to determine an appropriate user, because the operator may change to another doctor during the surgery.

In view of this, in the present technology, one user satisfying a predetermined condition is identified from among user candidates by using information acquired before the start of surgery and information acquired during surgery, and execution of an operation based on the speech, the line-of-sight position, or the like of another user candidate is inhibited.

With this configuration, it is possible to execute only an operation based on the speech or line-of-sight of an appropriate user in a situation where speeches and line-of-sight positions of a plurality of user candidates involved in surgery can be detected. That is, in the operating room, one appropriate user among a plurality of user candidates in a clean area can perform an operation on a device in an unclean area using a non-contact user interface (UI).

### User Determination Processing

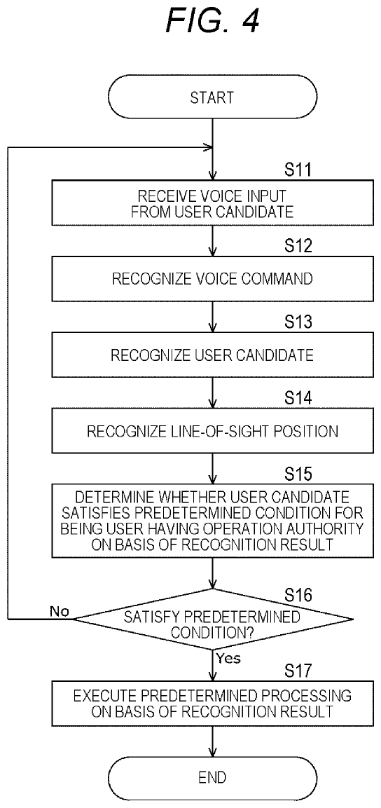

Next, a flow of user determination processing when voice input is used in a case where there are several user candidates in the operating room will be described with reference to a flowchart of FIG. 4.

When the user candidate speaks and a voice input from the user candidate is received by the microphone 50 (S11), the control unit 100 in the control device 10 executes processes of step S12 and subsequent steps.

In step S12, the recognition unit 111 analyzes the voice signal from the microphone 50, and recognizes a voice command included in the speech of the user candidate who has performed the voice input.

In step S13, the recognition unit 111 analyzes the voice signal from the microphone 50 and the image signal from the camera 60, and recognizes the user candidate who has performed the voice input. For example, in this recognition processing, it is recognized which user candidate in preset information corresponds to the user candidate that has performed the voice input.

In step S14, the recognition unit 111 analyzes the detection signal from the line-of-sight detector 70, and recognizes the line-of-sight position of the user candidate who has performed the voice input on the screen of the display device 40.

In step S15, the determination unit 112 determines whether or not the user candidate who has performed the voice input satisfies a predetermined condition for being a user having operation authority on the basis of the recognition result. The predetermined condition is determined using information acquired before the start of surgery, information acquired during surgery, and the like, which will be described in detail later.

When it is determined in step S15 that the predetermined condition is satisfied (“Yes” in S16), the processing proceeds to step S17. In step S17, the execution unit 113 executes predetermined processing on the basis of the recognition result.

Here, the user candidate determined to satisfy the predetermined condition, that is, the user identified from the plurality of user candidates, performs operation using voice and line-of-sight in combination.

For example, when a user gazing at a specific surgical site included in an operative field image displayed on the display device 40 issues a predetermined voice command, the execution unit 113 can enlarge and display a region of the specific surgical site to which the line-of-sight of the user is directed. In addition, when, for example, the user who is gazing at one thumbnail image among a plurality of thumbnail images displayed on the display device 40 issues a predetermined voice command, the execution unit 113 can display an image such as a reference image corresponding to the thumbnail image to which the line-of-sight of the user is directed.

Note that, although this example indicates the case where the operation is performed by a combination of voice and line-of-sight, the operation may be performed only by voice without using the line-of-sight of the user. In a case where the operation is performed only by voice, the process of step S14 may be skipped.

On the other hand, when it is determined in step S15 that the predetermined condition is not satisfied (“No” in S16), the processing returns to step S11, and the subsequent processes are repeated.

The flow of the user determination processing when voice input is performed has been described above. In this user determination processing, one user satisfying a predetermined condition is identified from the user candidates using information acquired before the start of surgery, or the like, and the identified user performs an operation using a non-contact user interface such as voice or line-of-sight. With this configuration, the execution of the operation by the speech, the line-of-sight position, or the like of other user candidates excluding the user candidate identified as the user is prevented, whereby an unintended operation can be prevented when devices are operated using voice input.

That is, when a first user registered in advance satisfies a predetermined condition in the imaging region in a case where surgery is performed using the medical display system 1, the display information displayed on the display device 40 is controlled on the basis of the voice of the first user or an input triggered by the voice. In addition, when the first user does not satisfy the predetermined condition in the imaging region, the display information is controlled on the basis of the voice of a second user or an input triggered by the voice. As a result, in a UI device that performs an operation while viewing a display unit using the voice, the line-of-sight, and the like of the user, only one user is determined, and only the operation by the voice, the line-of-sight, and the like of the user is executed.

Here, the imaging region includes a region (determination region) in which the face of the user candidate included in the captured image captured by the camera 60 can be determined. That is, the above wording “when the first user does not satisfy the predetermined condition in the imaging region” includes both meanings of “the first user is not in the imaging region” and “the first user is in the imaging region but is not in the determination region”.

### 2. Second Embodiment

As described above, the information acquired before the start of the surgery, the information acquired during the surgery, and the like are used as the predetermined condition used in the determination processing (S15 and S16 in FIG. 4) described above. More specifically, the predetermined condition can be determined on the basis of at least one of the information registered before the start of the surgery, information regarding a checking operation before the start of the surgery, or information regarding a specific situation during the surgery.

By setting the predetermined condition in this manner, it is possible to, for example, determine whether or not the information registered before the start of the surgery matches the recognition result of the user candidate who performs voice activation during the surgery, determine whether or not the situation of the user candidate during voice activation is appropriate as the user having operation authority, or determine whether or not the user candidate is on a predetermined position during voice activation.

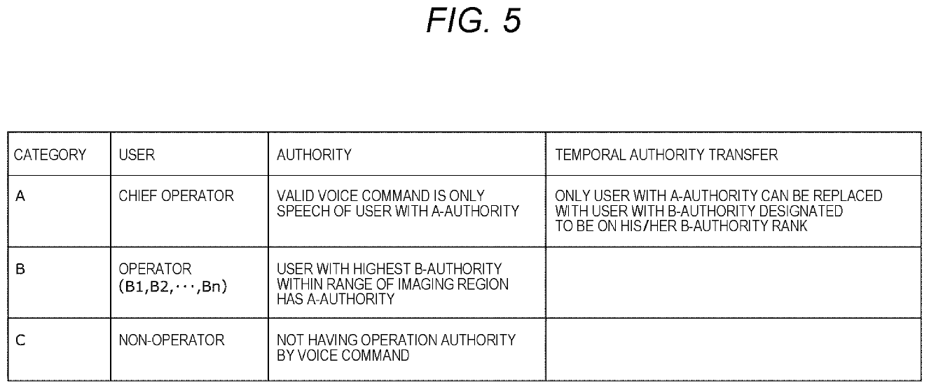

The user candidates can be classified into categories according to operation authority. FIG. 5 illustrates an example in which user candidates are classified into operation authorities of three categories A to C.

Category A corresponds to an operator (chief operator) who is a surgeon among surgical staffs in an operating room. Only the speech of the user candidate (user with A-authority) allocated to the authority of category A can be a valid voice command, and thus, the user with A-authority basically has the operation authority. However, it is possible to temporarily transfer the operation authority, and only the user having A-authority can be replaced with a user candidate (user with B-authority) designated to be on his/her B-authority rank.

Category B corresponds to a person who is directly involved in the surgical procedure and is likely to perform an operation on the display device 40, such as an operator such as an assistant or a scopist, among other surgical staffs. The user candidates (users with B-authority) allocated to the authority of category B have operation authority in a case where the user with A-authority is absent or cannot operate.

A B-authority rank corresponding to a number added to “B”, such as B1, B2, . . . , Bn (n: an integer of 1 or more), can be assigned to the user with B-authority. In this example, a smaller number indicates higher authority, and B1-authority is the highest. For example, when the operation authority is temporarily transferred, a user with the highest B-authority among the user candidates within the range of the imaging region can have A-authority.

Category C corresponds to a non-operator such as a nurse or a clinical engineer among other surgical staffs. The user candidates (users with C-authority) allocated to the authority of category C do not have the operation authority by the voice command.

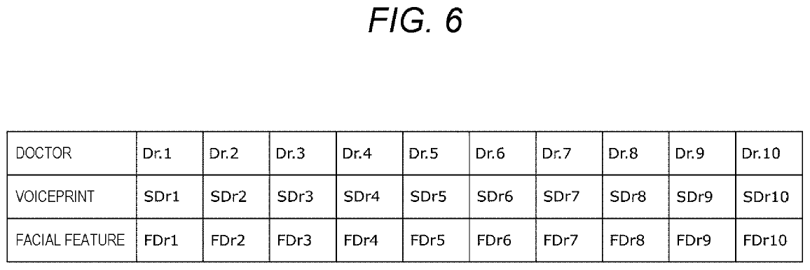

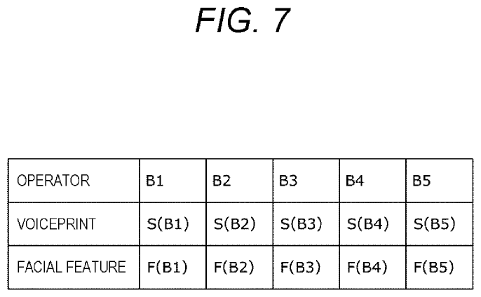

Furthermore, in a case where whether or not the user candidate is a user registered before the start of surgery is included as the predetermined condition, information such as voiceprints and facial features of the surgical staffs is stored in a database, and the information is assigned to each category before the start of surgery. Specifically, databases such as a doctor table illustrated in FIG. 6 and a preoperative registration table illustrated in FIG. 7 are used.

In FIG. 6, the doctor table stores information regarding a voiceprint and a facial feature for each piece of information for identifying a doctor. For example, SDr1 and FDr1 are registered as voiceprints and facial features of a doctor who is Dr. 1. Furthermore, voiceprints and facial features that are SDrx and FDrx are also registered for doctors Dr. 2 to Dr. 10.

In FIG. 7, the preoperative registration table stores information regarding a voiceprint and a facial feature for each piece of information regarding an operator (user candidate) registered before the start of surgery. That is, in the preoperative registration table (FIG. 7), the doctor data stored in the doctor table (FIG. 6) is assigned to operator data.

For example, in a case where the doctor Dr. 3 is assigned as an operator having B1-authority, SDr3 and FDr3 as the doctor data are assigned to the voiceprint S(B1) and F(B1) as the operator data.

Furthermore, in a case where the doctor Dr. 1 is assigned to an operator having B2-authority, SDr1 and FDr1 are assigned as the voiceprint S(B2) and F(B2), and in a case where the doctor Dr. 5 is assigned to an operator having B3-authority, SDr5 and FDr5 are assigned as the voiceprint S(B3) and F(B3).

Furthermore, in a case where the doctor Dr. 6 is assigned to an operator having B4-authority, SDr6 and FDr6 are assigned as the voiceprint S(B4) and F(B4), and in a case where the doctor Dr. 9 is assigned to an operator having B5-authority, SDr9 and FDr9 are assigned as the voiceprint S(B5) and F(B5).

In this manner, the doctor data stored in the doctor table (FIG. 6) is assigned to the operator data (data of the user with B-authority) stored in the preoperative registration table (FIG. 7) before the start of surgery, whereby it is possible to register face information used for identification of the B-authority rank and face recognition and voiceprint information used for speaker identification.

Note that, in order to prevent erroneous recognition of the user, the face information and the voiceprint information of a user having C-authority may be registered. These pieces of information can be used as information for determining a determination criterion of the user having A-authority.

Furthermore, in a case where whether or not the user candidate is a user candidate who has performed a checking operation (voice confirmation or the like) before the start of surgery is included as the predetermined condition, the information in the database is inquired at the time point at which the checking operation has been performed before the start of surgery, and the surgical staff who is the user candidate in the operating room is allocated to any one of categories A to C. As a result, the user candidate is classified as a user with A-authority, B-authority, or C-authority. The checking operation is performed not only at the time of entry into the operating room for the first time, but also at a predetermined timing such as when time-out occurs or when devices are operated.

In addition, information regarding the position (standing position or the like) of the user candidate may be used as the predetermined condition. The information regarding the position includes a position corresponding to the central portion of the screen of the display device 40 (for example, substantially the central portion in the width of the screen), a position corresponding to the central portion of the angle of view of the camera 60 mounted on the display device 40, and the like. That is, in most cases, an operator (chief operator) who is a surgeon stands at a position directly in front of the screen of the display device 40 and other staffs such as an assistant stands around the surgeon during an actual surgery, and thus, the positional relationship of them can be used.

Note that the above-described predetermined condition is an example, and other conditions may be set as long as they are set using information acquired before the start of surgery, information acquired during surgery, and the like. For example, the determination can be performed by determining, as the predetermined condition, whether or not the surgical procedure of the user candidate (the type of the surgical instrument the user candidate handles, or the like) corresponds to a predetermined surgical procedure.

In addition, as a method for recognizing the user candidate described above, image processing such as face recognition or bone recognition can be performed using an image signal from the camera 60, or voice processing such as voiceprint recognition or voice arrival direction recognition can be performed using a voice signal from the microphone 50. Known techniques can be used for techniques related to face recognition and bone recognition, and techniques related to voiceprint recognition and voice arrival direction recognition.

### Specific Example of User Determination

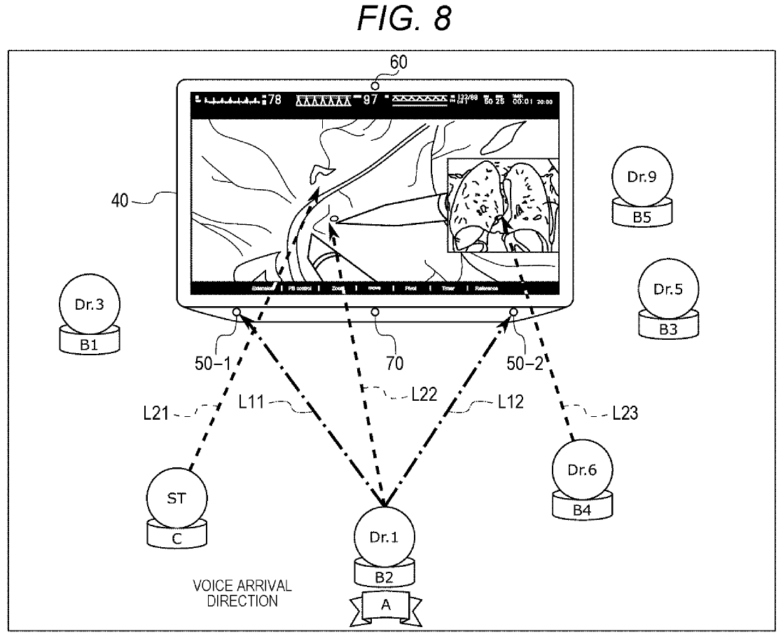

FIG. 8 illustrates a specific example of user determination during surgery.

In FIG. 8, six circles around the screen of the display device 40 represent user candidates, and the character written in each circle indicates a surgical staff such as a doctor. In addition, the character written in a column below each circle indicates the authority of each user candidate.

In FIG. 8, the doctor Dr. 3 to which B1-authority is assigned, the doctor Dr. 5 to which B3-authority is assigned, and the doctor Dr. 9 to which B5-authority is assigned are outside the imaging region. On the other hand, the doctor Dr. 1 to which B2-authority is assigned and the doctor Dr. 6 to which B4-authority is assigned are within the imaging region. In addition, another surgical staff ST such as a nurse is within the imaging region and is assigned with C-authority.

In this situation, it is assumed that the doctor Dr. 1 issues a voice command. In this case, the surgical staffs in the imaging region are imaged by the camera 60. An image signal obtained by the image capture is analyzed, by which three persons, the doctor Dr. 1, the doctor Dr. 6, and another surgical staff ST, are identified from the facial features.

When there is neither doctor (user) with A-authority nor doctor Dr. 3 assigned with B1-authority within the imaging region as described above, the doctor Dr. 1 assigned with B2-authority within the imaging region has A-authority. That is, in this example, the doctor with A-authority is replaced with the doctor Dr. 1 who is the doctor with B-authority which is the highest authority in the range of the imaging region among the doctors designated to be on his/her B-authority rank. The speech by the doctor Dr. 1 is collected by the microphones 50-1 and 50-2 as indicated by dash-dot-dash lines L11 and L12 in FIG. 8. The voiceprint S(IN_Dr1) of the voice command obtained by analyzing the voice signal matches the voiceprint S(B2) registered in the preoperative registration table.

Furthermore, the facial feature F(IN_ST) of the surgical staff ST in the voice arrival direction does not match the facial feature F(B2) registered in the preoperative registration table, whereas the facial feature F(IN_Dr1) of the doctor Dr. 1 matches the facial feature F(B2) registered in the preoperative registration table.

From these determination results, predetermined processing according to the voice command issued from the doctor Dr. 1 is executed on the basis of the line-of-sight information of the doctor Dr. 1 detected by the line-of-sight detector 70. For example, among the lines-of-sight of the user candidates in the imaging region indicated by broken lines L21 to L23 in FIG. 8, the broken line L22 indicates the line-of-sight of the doctor Dr. 1, and thus, the predetermined processing according to the voice command issued from the doctor Dr. 1 is executed for a specific surgical site to which the line-of-sight of the doctor Dr. 1 is directed in the visual field image.

### Voice-Enabled Processing

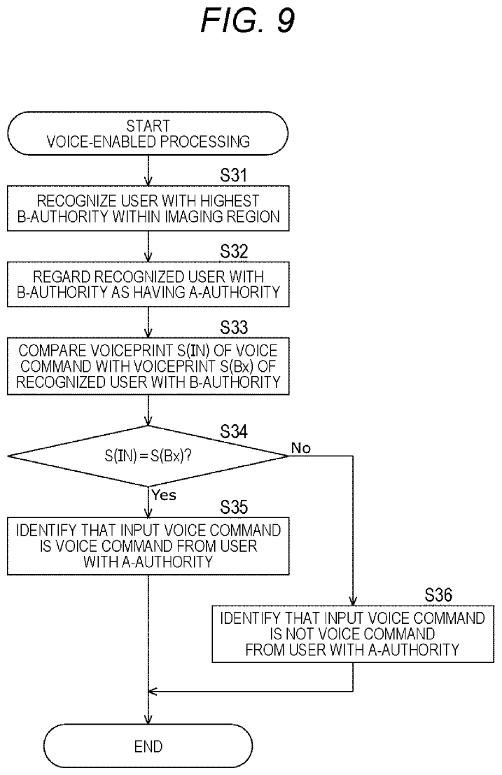

Here, the flow of voice-enabled processing applicable to the user determination illustrated in FIG. 8 will be described with reference to the flowchart of FIG. 9.

The recognition unit 111 recognizes the user with the highest B-authority from among the user candidates within the imaging region (S31), and regards the recognized user with the B-authority as having A-authority (S32). In the example of FIG. 8, the doctor Dr. 1 assigned with B2-authority within the imaging region has A-authority.

The determination unit 112 compares the voiceprint S(IN) of the input voice command with voiceprint S(Bx) of the recognized user having B-authority (S33).

When it is determined that the voiceprint comparison result satisfies S(IN)=S(Bx) (“Yes” in S34), the processing proceeds to step S35. Then, the execution unit 113 identifies that the input voice command is a voice command by the user having A-authority (S35), and executes predetermined processing according to the voice command. In the example of FIG. 8, the voice command issued from the doctor Dr. 1 is identified as a voice command from the user having A-authority, and predetermined processing is executed.

In addition, when it is determined that the voiceprint comparison result does not satisfy S(IN)=S(Bx) (“No” in S34), the processing proceeds to step S36. In this case, the execution unit 113 identifies that the input voice command is not a voice command by the user having A-authority (S36), and does not execute the voice command. In the example of FIG. 8, it is identified that the speech (voice command) by the doctor Dr. 6 or another surgical staff ST is not a voice command from the user having A-authority.

When the process of step S35 or S36 ends, the processing ends.

### Line-of-Sight-Enabled Processing

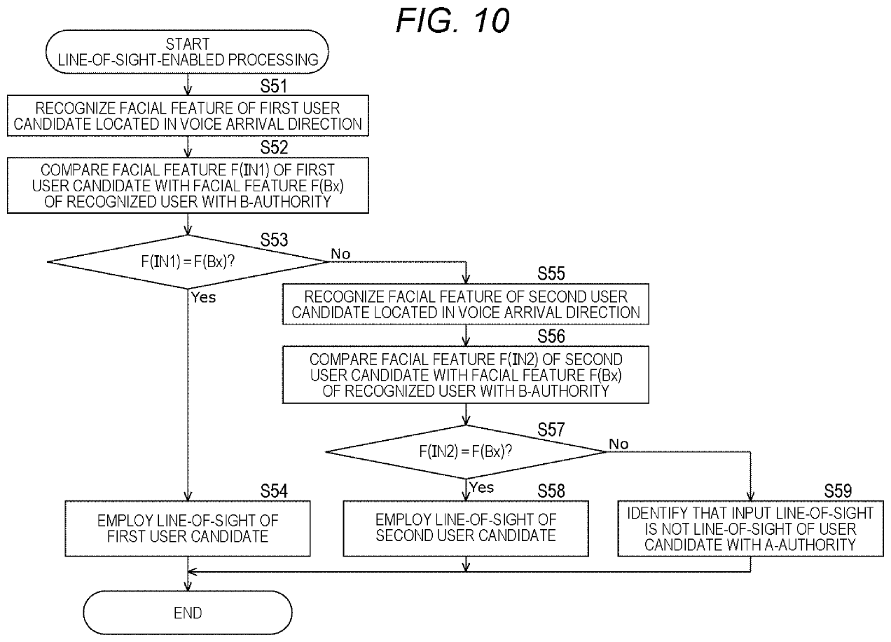

Next, the flow of line-of-sight-enabled processing applicable to the user determination illustrated in FIG. 8 will be described with reference to the flowchart of FIG. 10.

The recognition unit 111 recognizes the facial feature of a first user candidate located in the voice arrival direction (S51). The determination unit 112 compares the facial feature F(IN1) of the first user candidate with facial feature F(Bx) of the recognized user having B-authority (S52).

Note that the user with B-authority recognized in step S52 is the same as the user with B-authority recognized as the user with the highest B-authority within the range of the imaging region by the process of step S31 in FIG. 9.

When it is determined that the facial feature comparison result satisfies F(IN1)=F(Bx) by the process of step S52 (“Yes” in S53), the processing proceeds to step S54. Then, the execution unit 113 employs the line-of-sight of the first user candidate identified as the user (S54), and executes predetermined processing using the voice and the line-of-sight of the first user candidate.

In addition, when it is determined that the facial feature comparison result does not satisfy F(IN1)=F(Bx) by the process of step S52 (“No” in S53), the processing proceeds to step S55. In this case, the recognition unit 111 recognizes the facial feature of a second user candidate located in the voice arrival direction (S55).

The determination unit 112 compares the facial feature F(IN2) of the second user candidate with facial feature F(Bx) of the recognized user having B-authority (S56). Note that, here as well, the recognized user with B-authority means the user with the highest B-authority within the range of the imaging region.

When it is determined that the facial feature comparison result satisfies F(IN2)=F(Bx) by the process of step S57 (“Yes” in S57), the processing proceeds to step S58. Then, the execution unit 113 employs the line-of-sight of the second user candidate identified as the user (S58), and executes predetermined processing using the voice and the line-of-sight of the second user candidate.

In addition, when it is determined that the facial feature comparison result does not satisfy F(IN2)=F(Bx) by the process of step S57 (“No” in S57), the processing proceeds to step S59. In this case, the execution unit 113 identifies that the input voice command is not a voice command by the user having A-authority (S59), and the predetermined processing using voice and line-of-sight is skipped.

When the process of step S54, S58, or S59 ends, the processing ends.

Note that, although this examples indicates, for convenience of description, a case where there are faces of two user candidates, the first user candidate and the second user candidate, in the voice arrival direction, it is only required to compare facial features of all the user candidates in a case where there are faces of other user candidates.

### 3. Third Embodiment

In a case where there is a plurality of user candidates satisfying a predetermined condition, a priority order (rank) of the operation authority is determined for each user candidate, by which it is possible to specify a user who can perform operation according to the rank. As a method of ranking, various methods can be used. Rank information regarding the rank can be set before the start of surgery, can be changed according to a situation during surgery, or can be changed by being designated by a user (for example, a nurse) having specific authority.

### User Determination Processing According to Rank

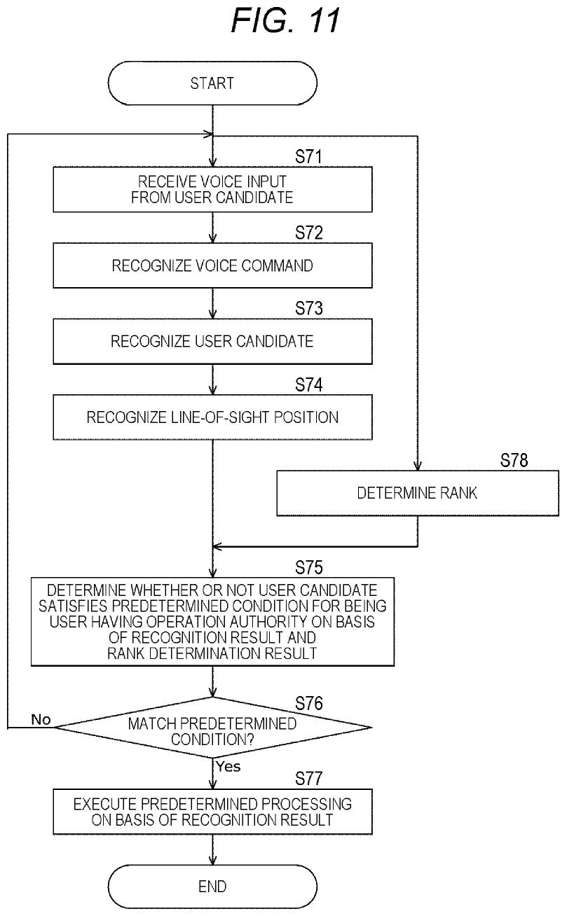

A flow of the user determination processing according to rank will be described with reference to the flowchart of FIG. 11.

In steps S71 to S74, a recognition result of the voice command, a recognition result of the user candidate, and a recognition result of the line-of-sight position are obtained as in steps S11 to S14 in FIG. 4. Furthermore, in step S78, the determination unit 112 determines the rank of the user candidate on the basis of the rank information. Details of the rank determination processing will be described later.

In step S75, the determination unit 112 determines whether or not the user candidate satisfies a predetermined condition for being a user having operation authority on the basis of the recognition result and the rank determination result.

When it is determined in step S75 that the predetermined condition is satisfied (“Yes” in S76), the processing proceeds to step S77. In step S77, the execution unit 113 executes predetermined processing on the basis of the recognition result.

In addition, when it is determined in step S75 that the predetermined condition is not satisfied (“No” in S76), the processing returns to step S71, and the subsequent processes are repeated.

The flow of the user determination processing using rank has been described above. Here, in the above-described rank determination processing (S78 in FIG. 11), the determination unit 112 determines the rank of the user candidate using the rank information by, for example, the following processing.

That is, the determination unit 112 acquires the rank information set for each user candidate, and determines the user candidate having the highest rank among the user candidates within the imaging region as the user on the basis of the rank of each user candidate.

Then, the execution unit 113 controls the display information displayed on the display device 40 on the basis of the voice and the line-of-sight of the user having the highest rank according to the determination result. For example, when the condition that the first user candidate is within the imaging region and the rank of the first user candidate is the highest is satisfied, operation based on the voice and the line-of-sight of the first user candidate as the user is received.

Furthermore, the determination unit 112 acquires rank information regarding a rank allocated in advance for each user candidate, and performs user determination by adjusting the rank allocated for each user candidate on the basis of information such as a checking operation before the start of surgery and the position of the user candidate.

For example, the user candidate who has issued a voice related to the checking operation before the start of the surgery can be set to the highest rank. Furthermore, when a user candidate having a higher rank than the preset rank (predefined rank) of the user candidate having the highest rank at the present time appears in the imaging region, the rank can be adjusted on the basis of the position information of the user candidate. That is, the detail of the information registered before the start of the surgery is set as an initial state, and the rank of the operation authority can be updated according to the situation of the surgery.

Note that, in a case where a predetermined operation is performed by a specific user, such as pressing of a reset button, the adjusted rank setting of the user candidate may be returned to a preset value.

In addition, in a case where the first user candidate moves from a position corresponding to the central portion of the screen of the display device 40 (for example, a substantially central portion in the horizontal width of the screen) or a position corresponding to the central portion of the angle of view of the camera 60, and the second user candidate is positioned in the vicinity of the central portion, the determination unit 112 performs the user determination to receive the voice activation of the second user candidate when the preset rank of the second user candidate is higher than that of the first user candidate. Furthermore, in this case, when the preset rank of the second user candidate is lower than that of the first user candidate, the user determination is performed so as to receive the voice activation of the first user candidate.

Note that, in a case where the registered user candidate is not within the imaging region, the user determination may be performed so as to receive voice activation of the user candidate at a position corresponding to the central portion of the screen of the display device 40 or the central portion of the angle of view of the camera 60.

In addition, the determination unit 112 performs user determination by regarding a user candidate designated by a specific user such as a nurse as the user candidate with the highest rank. In addition, who serves as the operator is registered in advance, by which the determination unit 112 can perform user determination as to who the operator is. For example, while the operator is present, the rank adjustment is restricted so that the rank cannot be switched.

Furthermore, the determination unit 112 can set a user candidate having a surgical tool, a user candidate having an electric scalpel, or a user candidate who views the screen of the display device 40 longer than a predetermined time to the highest rank on the basis of the image recognition result using the image signal from the camera 60 and the detection signal from the line-of-sight detector 70.

As described above, in a case where there is a plurality of user candidates satisfying the predetermined condition, operation by the speech and the line-of-sight of an appropriate user can be executed by determining a priority order (rank) of the operation authority. In addition, the priority order (rank) of the operation authority is determined on the basis of information set in advance, but the priority order may be changed in accordance with the situation of the surgery, and thus, the voice activation by a user more suitable for the situation can be executed.

### 4. Fourth Embodiment

As described above, the user with A-authority can temporarily transfer the authority to the user with B-authority. Such authority transfer may be performed when the user with A-authority issues a voice command (hereinafter referred to as a swap command) for user change, by which the authority is transferred to a designated user candidate (for example, the user with B-authority).

In addition, regarding cancel of the authority transfer, the authority transfer is canceled when the user with A-authority issues a command (hereinafter referred to as a swap cancel command) for canceling the user change, and the operation authority is returned from the designated user candidate (for example, the user with B-authority) to the user with A-authority.

### Authority Transfer Processing

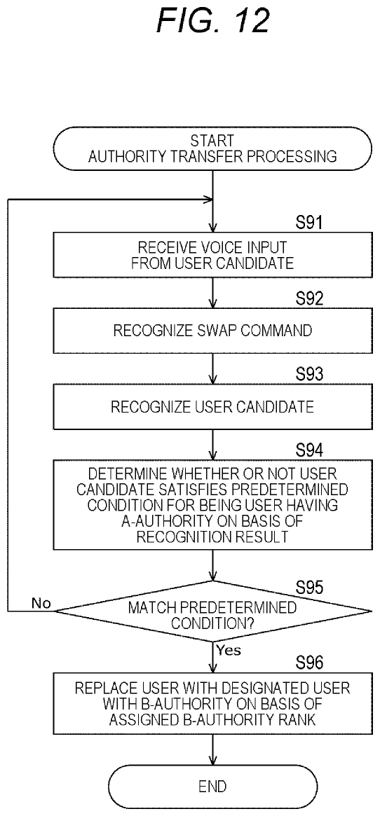

First, a flow of the authority transfer processing will be described with reference to the flowchart of FIG. 12.

When receiving a voice input from the user candidate (S91), the control device 10 executes processes of step S92 and the subsequent steps by the control unit 100.

In step S92, the recognition unit 111 analyzes the voice signal corresponding to the speech of the user candidate who has performed the voice input, and recognizes a swap command included in the speech of the user candidate. Here, a user candidate (user with B-authority) to which the authority is to be transferred is also designated, and this designation is also recognized.

In step S93, the recognition unit 111 analyzes an image signal obtained by imaging the user candidate that has performed the voice input, and recognizes the user candidate who has spoken.

In step S94, the determination unit 112 determines whether or not the user candidate who has performed the voice input satisfies a predetermined condition for being a user having A-authority on the basis of the recognition result.

When it is determined in step S94 that the predetermined condition is satisfied (“Yes” in S95), the processing proceeds to step S96. In step S96, the execution unit 113 replaces the user with A-authority with the designated user with B-authority (the user candidate to which the authority is to be transferred) on the basis of the B-authority rank assigned to the user with A-authority (the user candidate who has performed the voice input). As a result, the authority is temporarily transferred to the designated user with B-authority.

In addition, when it is determined in step S94 that the predetermined condition is not satisfied (“No” in S95), the processing returns to step S91, and the subsequent processes are repeated.

When the process of step S96 ends, the processing ends. The flow of the authority transfer processing has been described above.

### Authority Transfer Cancellation Processing

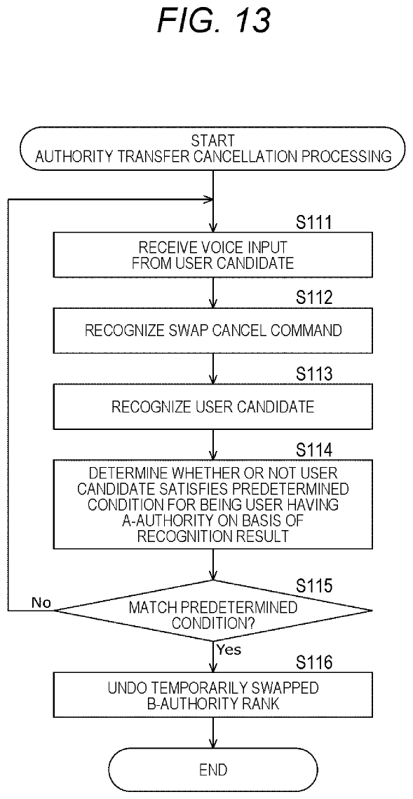

Next, a flow of the authority transfer cancellation processing will be described with reference to the flowchart of FIG. 13. However, it is assumed that, prior to the execution of the authority transfer cancellation processing illustrated in FIG. 13, the authority transfer processing (FIG. 12) described above is executed, and the authority has been transferred from the user with A-authority to the user with B-authority (user candidate to which the authority has been transferred).

When receiving a voice input from the user candidate (S111), the control device 10 executes processes of step S112 and the subsequent steps by the control unit 100.

In step S112, the recognition unit 111 analyzes the voice signal corresponding to the speech of the user candidate who has performed the voice input, and recognizes a swap cancel command included in the speech of the user candidate.

In step S113, the recognition unit 111 analyzes an image signal obtained by imaging the user candidate that has performed the voice input, and recognizes the user candidate who has spoken.

In step S114, the determination unit 112 determines whether or not the user candidate who has performed the voice input satisfies a predetermined condition for being a user having A-authority on the basis of the recognition result.

When it is determined in step S114 that the predetermined condition is satisfied (“Yes” in S115), the processing proceeds to step S116. In step S116, the execution unit 113 undoes the B-authority rank temporarily swapped to the designated user with B-authority (user candidate to which the authority has been transferred). As a result, the authority transfer is canceled, and the operation authority is returned from the designated user with B-authority (user candidate to which the authority has been transferred) to the user with A-authority.

In addition, when it is determined in step S114 that the predetermined condition is not satisfied (“No” in S115), the processing returns to step S111, and the subsequent processes are repeated.

When the process of step S116 ends, the processing ends. The flow of the authority transfer cancellation processing has been described above.

As described above, the operation authority can be temporarily transferred to the user candidate such as the user having B-authority regardless of the predetermined condition, whereby more flexible operation can be performed.

### 5. Fifth Embodiment

As described above, the user having C-authority does not have the operation authority by a voice command. A person who definitely cannot be a user can be excluded from the user candidates by performing user determination processing.

### User Exclusion Processing

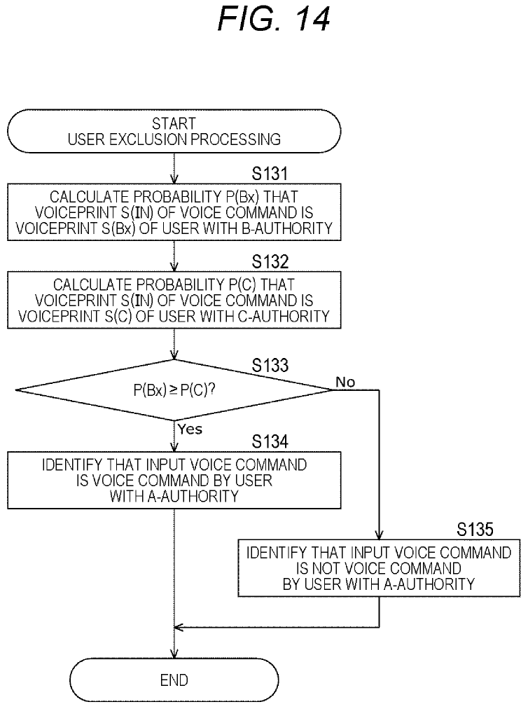

FIG. 14 is a flowchart for describing the flow of user exclusion processing.

In the user exclusion processing, when the voiceprint S(IN) of the voice command is determined through comparison in steps S33 and S34 in the above-described voice-enabled processing (FIG. 9), the user with C-authority is excluded from the user candidates in consideration of not only the voiceprint S(Bx) of the user with B-authority but also the voiceprint S(C) of the user with C-authority. With this processing, the accuracy of the user determination can be improved.

In step S131, the determination unit 112 calculates a probability P(Bx) that the voiceprint S(IN) of the voice command is the voiceprint S(Bx) of a user having B-authority.

In step S132, the determination unit 112 calculates a probability P(C) that the voiceprint S(IN) of the voice command is the voiceprint S(C) of a user having C-authority.

In step S133, the determination unit 112 compares the calculated value of the probability P(Bx) with the value of the probability P(C), and determines whether or not the value of the probability P(Bx) is equal to or greater than the value of the probability P(C).

When it is determined that P(Bx) P(C) is satisfied in the determination process of step S133, the processing proceeds to step S134. Then, the execution unit 113 identifies that the input voice command is a voice command by the user having A-authority (S134), and executes predetermined processing according to the voice command.

In addition, when it is determined that P(Bx)<P(C) is satisfied in the determination process of step S133, the processing proceeds to step S135. In this case, the execution unit 113 identifies that the input voice command is not a voice command by the user having A-authority (S135), and does not execute the voice command.

When the process of step S134 or S135 ends, the processing ends. The flow of the user exclusion processing has been described above.

Note that, although, in the above-mentioned user exclusion processing, the voiceprint S(C) of the user having C-authority is also considered when the voiceprint S(IN) of the voice command is determined through comparison in steps S33 and S34 in the above-described voice-enabled processing (FIG. 9), the user exclusion processing can also be applied to another processing.

For example, in the line-of-sight-enabled processing (FIG. 10) described above, a user with C-authority may be excluded from the user candidates by considering the facial feature F(C) of the user with C-authority as well as the facial feature F(Bx) of the user with B-authority when the facial features F(IN1) and F(IN2) are determined through comparison in steps S52 and S52 or steps S56 and S57.

As described above, a person who definitely cannot be a user can be excluded from the user candidates by comparing the data of the user with C-authority as well as the data of the user with B-authority during determination of the voiceprint S(IN) of the voice command in the voice-enabled processing (FIG. 9) or during the determination of the facial feature F(IN) in the line-of-sight-enabled processing (FIG. 10). As a result, the accuracy of the user determination can be further improved, and the reliability in identifying the user can be improved.

### Configuration Example of Computer

A program executed by the (control unit 100 of) the control device 10 may be a program in which processes are carried out in time series in the order described in this specification or may be a program in which processes are carried out in parallel or at necessary timings, such as when the processes are called.

The series of processing described above can be executed by hardware or by software. In a case where the series of processing is executed by software, a program constituting the software is installed to a computer incorporated in dedicated hardware, a general-purpose personal computer, or the like.

The program to be installed is provided by being recorded in a removable recording medium including an optical disk (compact disc-read only memory (CD-ROM), digital versatile disc (DVD), and the like), a semiconductor memory, and the like. In addition, the program may also be provided via a wired or wireless transmission medium such as a local area network, the Internet, and digital broadcasting. The program can be pre-installed in a ROM or a recording unit.

Note that the program executed by the computer may be a program in which processes are carried out in time series in the order described in this specification or may be a program in which processes are carried out in parallel or at necessary timings, such as when the processes are called.

Note that, in the present specification, a “system” means a set of a plurality of components (devices, modules (parts), etc.), and all constituent elements need not be necessarily in the same housing. Therefore, a plurality of devices housed in separate housings and connected via a network, and a single device in which a plurality of modules are housed in one housing are regarded as a system.

It should be noted that the effects described in the present specification are merely illustrative and not restrictive, and may have additional effects.

It should be noted that embodiments of the present technology are not limited to the abovementioned embodiments, and various modifications are possible without departing from the gist of the present technology.

The present technology can employ, for example, a configuration of cloud computing in which one function is shared by a plurality of apparatuses via a network and processed in cooperation with each other.

Further, steps described in the above-described flowcharts may be executed by a single device or shared and executed by a plurality of devices. Further, in a case where multiple processes are included in one step, the multiple processes included in the one step may be executed by a single device or may be shared and executed by a plurality of devices.

It is to be noted that the present technology may also have the following configurations.

(1)

A medical display system including:

a display unit that provides a display on the basis of information output from a medical device;

an imaging unit that images, as an imaging region, a part of a region from which at least the display unit is viewable;

a voice acquisition unit that acquires a voice in the region from which at least the display unit is viewable; and

a control unit that controls display information on the basis of the information output from the medical device, in which

the control unit
controls, when a first user registered in advance satisfies a predetermined condition in the imaging region, the display information on the basis of a voice of the first user or an input triggered by the voice, and
controls, when the first user does not satisfy the predetermined condition in the imaging region, the display information on the basis of a voice of a second user different from the first user or an input triggered by the voice.
   (2)

The medical display system according to (1), in which

the control unit determines the predetermined condition on the basis of at least one of information registered before a start of a surgery, information regarding a checking operation before the start of the surgery, or information regarding a specific situation during the surgery.

(3)

The medical display system according to (2), in which

the control unit
recognizes a user candidate including the first user and the second user on the basis of at least one of an image captured by the imaging unit or a voice acquired by the voice acquisition unit, and
determines whether or not the user candidate that has been recognized satisfies the predetermined condition.
   (4)

The medical display system according to (3), in which

the information registered before the start of the surgery includes information regarding the user candidate.

(5)

The medical display system according to (4), in which

the information regarding the user candidate includes correspondence information in which information regarding a feature of an operator is associated with information regarding a feature of a surgeon.

(6)

The medical display system according to (3) or (4), in which

the information regarding the checking operation before the start of the surgery includes information regarding an operation authority including a plurality of categories, and

the control unit allocates the user candidate who has performed the checking operation before the start of the surgery into the categories of the operation authority.

(7)

The medical display system according to (6), in which

the categories of the operation authority include a first category which has the operation authority and in which a voice command is valid, a second category in which the voice command is valid when the operation authority is transferred, and a third category not having the operation authority.

(8)

The medical display system according to (3), (4), or (6), in which

the information regarding a specific situation during the surgery includes information regarding a position of the user candidate.

(9)

The medical display system according to (8), in which

the information regarding the position of the user candidate includes a position corresponding to a substantially central portion of the display unit in a horizontal width.

(10)

The medical display system according to (3), (4), (6), or (8), in which,

in a case where there is a plurality of user candidates satisfying the predetermined condition, the control unit sets a priority order of the operation authority for each of the user candidates.

(11)

The medical display system according to (10), in which

the control unit updates the priority order of the operation authority according to a situation of the surgery with details of the information registered before the start of the surgery as an initial state.

(12)

The medical display system according to (10) or (11), in which

the control unit updates the priority order of the operation authority according to an operation performed by a user having a specific authority.

(13)

The medical display system according to any one of (3) to (12), in which

the control unit temporarily transfers the operation authority to a designated user candidate when the first user satisfying the predetermined condition issues a voice command for user change.

(14)

The medical display system according to (13), in which

the control unit returns the operation authority to the first user from the designated user candidate when the first user satisfying the predetermined condition issues a voice command for canceling the user change.

(15)

The medical display system according to any one of (3) to (12), in which

the control unit performs recognition processing for a user candidate that is not going to be the first user and the second user, and excludes the user candidate that is not going to be the first user and the second user from the user candidate.

(16)

The medical display system according to any one of (1) to (15), in which

the input triggered by the voice includes a line-of-sight of the first user or the second user when the first user or the second user issues a voice command.

(17)

The medical display system according to (16), in which

the control unit executes predetermined processing according to the voice command on the basis of a line-of-sight position of the first user or the second user on the display unit.

(18)

The medical display system according to (17), further including

a line-of-sight detection unit that detects the line-of-sight of the first user or the second user within the imaging region.

(19)

A control method performed when a medical display system controls display information displayed on a display unit on the basis of information output from a medical device, the control method including:

controlling, when a first user registered in advance satisfies a predetermined condition in an imaging region that is imaged as a part of a region from which at least the display unit is viewable, the display information on the basis of a voice of the first user acquired in the region from which at least the display unit is viewable or an input triggered by the voice; and

controlling, when the first user does not satisfy the predetermined condition in the imaging region, the display information on the basis of a voice of a second user acquired in the region from which at least the display unit is viewable or an input triggered by the voice, the second user being different from the first user.

(20)

A control device including

a control unit that controls display information displayed on a display unit on the basis of information output from a medical device, in which

the control unit
controls, when a first user registered in advance satisfies a predetermined condition in an imaging region that is imaged as a part of a region from which at least the display unit is viewable, the display information on the basis of a voice of the first user acquired in the region from which at least the display unit is viewable or an input triggered by the voice, and
controls, when the first user does not satisfy the predetermined condition in the imaging region, the display information on the basis of a voice of a second user acquired in the region from which at least the display unit is viewable or an input triggered by the voice, the second user being different from the first user.

### REFERENCE SIGNS LIST

1 Medical display system

10 Control device

20 Microscope device

30 Monitoring device

40 Display device (display unit)

50, 50-1, 50-2 Microphone (voice acquisition unit)

60 Camera (imaging unit)

70 Line-of-sight detector (line-of-sight detection unit)

100 Control unit

111 Recognition unit

112 Determination unit

113 Execution unit

1. A medical display system comprising:
a display unit that provides a display on a basis of information output from a medical device;
an imaging unit that images, as an imaging region, a part of a region from which at least the display unit is viewable;
a voice acquisition unit that acquires a voice in the region from which at least the display unit is viewable; and
a control unit that controls display information on a basis of the information output from the medical device, wherein
the control unit
controls, when a first user registered in advance satisfies a predetermined condition in the imaging region, the display information on a basis of a voice of the first user or an input triggered by the voice, and
controls, when the first user does not satisfy the predetermined condition in the imaging region, the display information on a basis of a voice of a second user different from the first user or an input triggered by the voice.

2. The medical display system according to claim 1, wherein
the control unit determines the predetermined condition on a basis of at least one of information registered before a start of a surgery, information regarding a checking operation before the start of the surgery, or information regarding a specific situation during the surgery.

3. The medical display system according to claim 2, wherein
the control unit
recognizes a user candidate including the first user and the second user on a basis of at least one of an image captured by the imaging unit or a voice acquired by the voice acquisition unit, and
determines whether or not the user candidate that has been recognized satisfies the predetermined condition.

4. The medical display system according to claim 3, wherein
the information registered before the start of the surgery includes information regarding the user candidate.

5. The medical display system according to claim 4, wherein
the information regarding the user candidate includes correspondence information in which information regarding a feature of an operator is associated with information regarding a feature of a surgeon.

6. The medical display system according to claim 3, wherein
the information regarding the checking operation before the start of the surgery includes information regarding an operation authority including a plurality of categories, and
the control unit allocates the user candidate who has performed the checking operation before the start of the surgery into the categories of the operation authority.

7. The medical display system according to claim 6, wherein
the categories of the operation authority include a first category which has the operation authority and in which a voice command is valid, a second category in which the voice command is valid when the operation authority is transferred, and a third category not having the operation authority.

8. The medical display system according to claim 3, wherein
the information regarding a specific situation during the surgery includes information regarding a position of the user candidate.

9. The medical display system according to claim 8, wherein
the information regarding the position of the user candidate includes a position corresponding to a substantially central portion of the display unit in a horizontal width.

10. The medical display system according to claim 3, wherein,
in a case where there is a plurality of user candidates satisfying the predetermined condition, the control unit sets a priority order of the operation authority for each of the user candidates.

11. The medical display system according to claim 10, wherein
the control unit updates the priority order of the operation authority according to a situation of the surgery with details of the information registered before the start of the surgery as an initial state.

12. The medical display system according to claim 10, wherein
the control unit updates the priority order of the operation authority according to an operation performed by a user having a specific authority.

13. The medical display system according to claim 3, wherein
the control unit temporarily transfers the operation authority to a designated user candidate when the first user satisfying the predetermined condition issues a voice command for user change.

14. The medical display system according to claim 13, wherein
the control unit returns the operation authority to the first user from the designated user candidate when the first user satisfying the predetermined condition issues a voice command for canceling the user change.

15. The medical display system according to claim 3, wherein
the control unit performs recognition processing for a user candidate that is not going to be the first user and the second user, and excludes the user candidate that is not going to be the first user and the second user from the user candidate.

16. The medical display system according to claim 1, wherein
the input triggered by the voice includes a line-of-sight of the first user or the second user when the first user or the second user issues a voice command.

17. The medical display system according to claim 16, wherein
the control unit executes predetermined processing according to the voice command on a basis of a line-of-sight position of the first user or the second user on the display unit.

18. The medical display system according to claim 17, further comprising
a line-of-sight detection unit that detects the line-of-sight of the first user or the second user within the imaging region.

19. A control method performed when a medical display system controls display information displayed on a display unit on a basis of information output from a medical device, the control method comprising:
controlling, when a first user registered in advance satisfies a predetermined condition in an imaging region that is imaged as a part of a region from which at least the display unit is viewable, the display information on a basis of a voice of the first user acquired in the region from which at least the display unit is viewable or an input triggered by the voice; and
controlling, when the first user does not satisfy the predetermined condition in the imaging region, the display information on a basis of a voice of a second user acquired in the region from which at least the display unit is viewable or an input triggered by the voice, the second user being different from the first user.

20. A control device comprising
a control unit that controls display information displayed on a display unit on a basis of information output from a medical device, wherein
the control unit
controls, when a first user registered in advance satisfies a predetermined condition in an imaging region that is imaged as a part of a region from which at least the display unit is viewable, the display information on a basis of a voice of the first user acquired in the region from which at least the display unit is viewable or an input triggered by the voice, and
controls, when the first user does not satisfy the predetermined condition in the imaging region, the display information on a basis of a voice of a second user acquired in the region from which at least the display unit is viewable or an input triggered by the voice, the second user being different from the first user.
